(19) United States
(12) Patent Application Publication
(10) Pub. No.: US 2026/0286411 A1
(43) Pub. Date: Sep. 24, 2026

(54) A PROCESS FOR SYNTHESIS OF BACTERIAL NANO-CELLULOSE USING ROTARY DISC BIOREACTOR

(71) Applicant: COUNCIL OF SCIENTIFIC & INDUSTRIAL RESEARCH AN INDIAN REGISTERED BODY INCORPORATED UNDER THE REGN., New Delhi, Delhi (IN)

(72) Inventors: Syed Gulam DASTAGER, PUNE (IN); Ashish Siddharth JAGTAP, PUNE (IN)

(21) Appl. No.: 19/476,389

(22) PCT Filed: Apr. 17, 2024
(86) PCT No.: PCT/IN2024/050404

§ 371 Date: Oct. 17, 2025

(30) Foreign Application Priority Data
Apr. 20, 2023 (IN) 202311028953

(51) Int. Cl. C12P 19/04; B82Y 30/00; B82Y 40/00; C12M 1/12; C12M 3/06; C12N 1/205; C12R 1/01

(52) U.S. Cl. CPC C12P 19/04; C12M 25/06; C12M 27/14; C12N 1/205; B82Y 30/00; B82Y 40/00; C12R 2001/01

(57) ABSTRACT

An efficient process for producing bacterial nanocellulose (BNC) in high yield and in a shorter duration of 5-10 days, by using a rotatory disc bioreactor. The bacterial strain used to achieve high concentration of BNC is K. rhaeticus MCC 0157.

### FIELD OF THE INVENTION

The present invention relates to a process for the production of bacterial nano cellulose (BNC). More particularly, the present invention relates to an efficient process for obtaining high yields of bacterial nanocellulose expeditiously, wherein the inoculation and fermentation is carried out in a rotatory disc bioreactor.

### BACKGROUND OF THE INVENTION

Bacterial nanocellulose (BNC), also known as microbial nanocellulose, is an extracellular macromolecule. BNC can be biodegraded by cellulase or microorganisms in the natural environment and would not cause pollution as usual plastic. BNC is mainly synthesized by using static cultivations of acetic acid bacteria to show isotropic morphology and properties.

Compared to plant nanocellulose, BNC has a much higher degree of polymerization (DP), superfine fiber diameter ranging from 20 to 80 nm, huge water-holding capacity, and high wet tensile strength. However, wider, and more innovative applications of the traditional BNC hydrogel membrane from static culture are restricted by its limited structure, single performance, and relatively high price in a dry state as compared to plant nanocellulose. The limitation of structure and properties of BNC hydrogel membrane is mainly due to the difficulty in regulating the structure during the traditional static cultivation and also in the post-processing. The high price of dry BNC could be mostly attributed to the low productivity and yield of BNC in slow-growing static bacterial cultures.

Although conventional static cultivation has been applied extensively for bio nanocellulose production, it suffers from low efficiency and high cost.

One of the approaches attempted to produce BNP is disclosed in an Indian Patent Application number 201911024544. The disclosed process of preparation of bacterial cellulose production uses Komagataeibacter rhaeticus where the cellulose production yield is in range of 5 to 10 grams/liter and cellulose has crystalline index in the range of 75-81%, and the process is carried out under static mode. However, this approach is not efficient as it requires at least 10-15 days to complete the process for achieving 5-10 grams/liter yield.

There are some reports which provide bacterial cellulose production under rotation mixing mode using rotary disc bioreactor e.g. Shin-Ping Lin et al., Cellulose (2014) 21:835-844; and Chhavi Sharma et al., Cellulose (2022) 29:7177-7191. However, the yields reported therein are lower considering the longer residence time and lesser effective absorption of discs used therein.

Thus, there is still an unmet need to arrive at an efficient process with enhanced production of bacterial nanocellulose, with high yield in shorter duration.

### OBJECTIVE OF THE INVENTION

An objective of the present invention is to provide a process for the production of bacterial nanocellulose.

Another objective of the present invention is to provide a process for the bacterial nanocellulose production with high yield.

Another objective of the present invention is to provide an efficient process for producing bacterial nanocellulose using a rotatory disc bioreactor.

Another objective of the present invention is to provide a rotatory disc bioreactor for efficient production of bacterial nanocellulose.

### SUMMARY OF THE INVENTION

The present invention generally relates to production of Bacterial Nanocellulose (BNC).

The present invention provides a process for production of bacterial nanocellulose (BNC), comprising the steps of:
a. inoculating bacteria Komagataeibacter rhaeticus MCC 0157 to a nutrient medium in a rotatory disc bioreactor and
b. fermenting at a speed of 5-15 RPM for a time period of 5-10 days,
   wherein the rotatory disc bioreactor comprises at least two circular rotatory discs of hydrophilic absorbent material placed at a predetermined distance of 2-3 inch between them.

Specifically, the present invention relates to an efficient process for production of bacterial nanocellulose by fermenting bacteria Komagataeibacter rhaeticus MCC 0157 in a nutrient medium at a specific pH, and under a specific rotation (RPM) for a period of 5-10 days, wherein the inoculation and fermentation are carried out in a rotatory disc bioreactor comprising at least two circular rotatory discs.

Accordingly, in an aspect, the present invention provides a process of production of bacterial nanocellulose comprising inoculating and fermenting bacterial species Komagataeibacter rhaeticus MCC 0157 in a nutrient medium at specific pH up to the range of 6-7, at a specific rotation RPM (Rotation per minutes) of 5-15 for a period of 5-10 days with monitoring the yield of the produced BNC at an interval of 2-5 days, wherein the inoculation and fermentation are carried out in a rotatory disc bioreactor comprising at least two circular rotatory discs placed at a predetermined distance of 2-3 inches, and said discs are dipped in said nutrient medium.

In an aspect, the present invention provides a process for production of bacterial nanocellulose (BNC), the process comprising the steps of: fermenting bacteria Komagataeibacter rhaeticus MCC 0157 in a nutrient medium at a speed in the range of 5-15 RPM for a time period in the range of 5-10 days, wherein fermenting the bacteria is carried out in a rotatory disc bioreactor comprising at least two circular rotatory discs placed at a predetermined distance in the range of 2-3 inches.

In an embodiment, wherein the nutrient medium is modified Hestrin-Schramm (HS) liquid medium comprising glycerol in the weight range of 2-3%; yeast extract in the weight range of 0.1-2.0%; peptone in the weight range of 0.1-2.0%; disodium hydrogen phosphate in the weight range of 0.1-1.00%; citric acid in the weight range of 0.1-1.0% and water in quantity sufficient with respect to the medium.

In specific embodiment, wherein the nutrient medium is modified Hestrin-Schramm (HS) liquid medium comprising glycerol in the weight range of 2-3%; yeast extract 0.5%; peptone 0.5%; disodium hydrogen phosphate 0.27%; citric acid 0.115% and water in quantity sufficient with respect to the medium.

In an embodiment, wherein the pH of the nutrient medium is maintained in the range of 6 to 7, or at neutral or near to neutral region.

In an embodiment, fermenting K. rhaeticus MCC 0157 in the nutrient medium is carried out at a speed selected from 5, 7, 10, or 15 RPM.

In an embodiment, wherein the circular rotatory discs of the bioreactor are immersed in the nutrient medium.

In an embodiment, wherein the production of bacterial nanocellulose (BNC) is monitored at an interval of 2 to 5 days and adding additional media in case of complete utilization of the media to monitor the yield of the BNC for subsequent days till 5 to 10 days.

In an embodiment, wherein the fermentation is carried out for 5 days.

In another aspect, the present invention provides a rotary disc bioreactor (RDB) (100) comprising:
a) a plurality of water absorbing disc (101) with pre-defined distance between them and arranged to be immersed in a nutrient medium (108),
b) a rod (103) for holding said discs (101),
c) a rotation motor (105) and a control panel (109) connected to the said rod (103), and
d) an outer chamber (107) for covering or protecting said rod and discs.

In an embodiment, wherein the water absorbing discs are made of hydrophilic absorbent material.

In an embodiment, wherein the water absorbing discs are made of wood.

In an embodiment, wherein the distance between the discs is 2-3 inches.

In another aspect, the process and rotary disc bioreactor disclosed herein provides production of bacterial nanocellulose with enhanced/improved yield of over 10 grams of dry weight, at around or within 5 days.

These and other features, aspects, and advantages of the present subject matter will be better understood with reference to the following description. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

This invention is illustrated with the help of the following accompanying drawings.

FIG. 1 is a schematic representation of design of Rotary disc bioreactor (RDB) (100), as per one of the exemplary embodiments of the present invention.

FIG. 2 shows: bacterial nanocellulose (BNC) pellicles (110) on (a) static and (b) RDB, as per one of the exemplary embodiments of the present invention.

FIGS. 3A and B provide graphs depicting the yield of BNC produced in RDB and under static condition on the basis of wet BNC (A) and dry BNC (B), as per one of the exemplary embodiments of the present invention.

FIG. 4 is a graph depicting: (a) yield of BNC on discs made of different materials, (b) optimization of rotational speed, (c) optimization of bacterial nanocellulose yield on varying days, and (d) optimization of distance between each disc, as per one of the exemplary embodiments of the present invention.

FIG. 5 is a graph showing yield of RDB produced on various media in the RDB and under the static condition, as per one of the exemplary embodiments of the present invention.

FIG. 6 are field emission scanning electron microscopy (FE-SEM) images revealing the fibrillar and non-woven arrangement of bacterial nanocellulose from RDB using different media, as per one of the exemplary embodiments of the present invention.

FIG. 7 is X-ray diffraction (XRD) spectra of membranes of BNC produced under static and RDB conditions. The XRD patterns obtained showing the characteristics peaks of cellulose at 2Θ angles z 14°, 16° and 22° in both the conditions, as per one of the exemplary embodiments of the present invention.

FIGS. 8A and 8B show CP/MAS ¹³C nuclear magnetic resonance (NMR) spectra of the bacterial nanocellulose grown under static cultivation and RDB.

FIGS. 9A and 9B are Fourier transform Infrared spectroscopy (FTIR) spectra of BNC grown in static vs. RDB conditions.

FIG. 10 shows thermogravimetric analysis (TGA) analysis of BNC grown in static vs. RDB conditions, as per one of the exemplary embodiments of the present invention.

### DETAILED DESCRIPTION OF THE INVENTION

The invention will now be described in detail in connection with certain preferred and optional embodiments, so that various aspects thereof may be more fully understood and appreciated.

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”.

“Including” and “including but not limited to” are used interchangeably.

The term “BC” or “BNC” as used herein refers to bacterial nanocellulose.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The microbe used in the present invention “Komagataeibacter rhaeticus” is already deposited with deposition authority National Centre for Cell Science (NCCS), Pune, Maharashtra, India on 3 Jan. 2019 and have been assigned with accession no. MCC 0157. It is noted that the microbes “Komagataeibacter rhaeticus” and “K. rhaeticus PG2” are used herein interchangeably bearing the same meaning throughout the specification.

The present invention relates to a process for enhancing the yield of bacterial nanocellulose. More particularly the present invention discloses a process employing bacteria K. rhaeticus for the production of bacterial nanocellulose. Accordingly, in an embodiment, the present invention provides a process for production of bacterial nanocellulose, the process comprising the steps of fermenting bacteria Komagataeibacter rhaeticus MCC 0157 in a nutrient medium at a speed in the range of 5-15 RPM for a time period in the range of 5-10 days with monitoring the yield of the produced BNC at an interval in the range of 2-5 days, wherein fermenting the bacteria is carried out in a rotatory disc bioreactor comprising at least two circular rotatory discs placed at a predetermined distance in the range of 2-3 inches.

In certain embodiment, the process can include a step of monitoring the yield of the produced BNC at an interval in the range of 2 to 5 days respectively for the fermentation period in the range of 2-10 days to ensure that the reaction/fermentation has not stopped and adding additional media in case of complete utilization of the media via fed batch approach.

In certain embodiment, the process for production of bacterial nanocellulose includes the steps of:
a) isolating bacteria Komagataeibacter rhaeticus; and
b) inoculating bacteria K. rhaeticus in a nutrient medium comprising of glycerol, yeast extract, peptone, disodium hydrogen phosphate, citric acid, and water to provide the inoculant Komagataeibacter rhaeticus MCC 0157.

The bacterial strain K. rhaeticus is isolated from rotten pomegranate.

In an embodiment, the isolated K. rhaeticus strain showed potential for the highest BNC production in the standard as well as modified Hestrin-Schramm (HS) liquid medium.

In an embodiment, the nutrient medium is standard HS medium. The composition of standard HS medium comprises glucose 2%; yeast extract 0.5%; peptone 0.5%; disodium hydrogen phosphate 0.27%; and citric acid 0.115%.

The standard HS media is modified by replacing the 2% glucose with 2-3% glycerol. In certain embodiments, glycerol may be 3%.

The modified HS media comprises glycerol in the weight range of 2-3%; yeast extract 0.5%; peptone 0.5%; disodium hydrogen phosphate 0.27%; and citric acid 0.115%.

In an embodiment, the nutrient medium employed is the modified HS media comprising glycerol in the weight range of 2-3%, yeast extract in an amount of 0.5%, peptone in an amount of 0.5%, disodium hydrogen phosphate in an amount of 0.27%, citric acid in an amount of 0.115%, and water in quantity sufficient

In an embodiment of the present invention, the pH of the nutrient medium is maintained in the range of 6 to 7, or at neutral or near to neutral region.

In an embodiment, the fermentation of a nutrient medium with K. rhaeticus MCC 0157 is carried out at a speed selected from 5, 7, 10, and 15 RPM.

In certain embodiments, fermenting K. rhaeticus MCC 0157 in the nutrient medium is carried out at a speed selected from 5, or 6, or 7, or 8, or 9, or 10, or 11, or 12, or 13, or 14, or 15 RPM.

In an embodiment, the distance between the two circular rotatory discs in the bioreactor is in the range of 2-3 inch.

In an embodiment, the distance between the two circular rotatory discs in the bioreactor is kept at 2 inch.

In an embodiment, the circular rotatory discs of the bioreactor are immersed in the nutrient medium.

In an embodiment, the fermentation is carried out for 5 days.

In certain embodiments, the process of the present invention optionally comprises filtering, washing and purifying the bacterial cellulose nanoparticles to obtain pure bacterial cellulose nanoparticles.

In accordance with the process of the present invention, wherein the fermentation is carried out in a rotary disc bioreactor, the organisms adhere to a rotating disc and remain entrapped in the fibrillar network to provide enhanced yield of BNC.

The process of the present invention not only enhances the yield of BNC but provides the high yield of over 10 gl⁻¹ dry weight, within 5 days of fermentation, thus proving the process of the present invention to be efficient as compared to the static cultivation, wherein even the highest yield is not so high, and it requires at least 15 days of fermentation to obtain the highest yield.

In another embodiment, the present invention provides a rotary disc bioreactor for enhancing the yield of bacterial nanocellulose, wherein the rotary disc bioreactor comprises at least two circular rotary discs, a rod having 2-4 mm, preferably 3 mm threading to mount the disc, an outer chamber which can maintain the sterility throughout the entire process, and the rotation motor or mechanistic motor having the control panel to control the speed.

In an embodiment, the present invention provides a rotary disc bioreactor (RDB) (100) comprising:
a) a plurality of water absorbing disc (101) with pre-defined distance between them and arranged to be immersed in a nutrient medium (108),
b) a rod (103) for holding said discs (101),
c) a rotation motor (105) and a control panel (109) connected via said rod (103) for attaining rotation mode for said rod (103) and discs (101) at specified rotational speed and time, and
d) an outer chamber (107) for covering or protecting said rod and discs.

The reactor disclosed herein may have any suitable dimension as per the scalability of the process to retain 1-50 L of media and grams to kilogram yield of BNC.

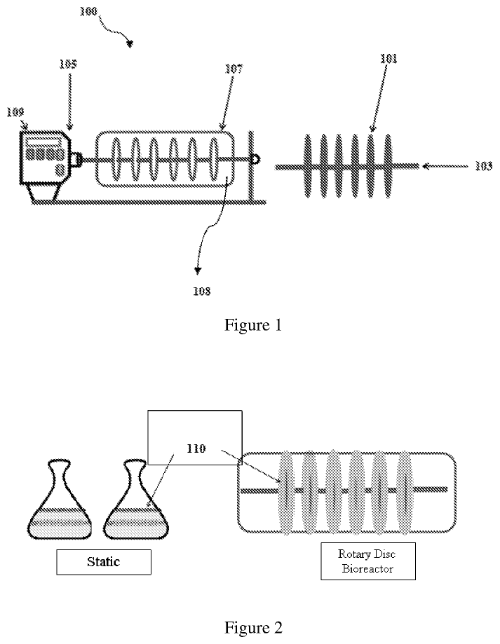

In a specific exemplary embodiment, referring to FIG. 1, the present invention provides a rotary disc bioreactor (RDB) (100) for 1 liter media comprising a dimension of the outer chamber (107) about 25-40×10-20×15-25 or about 32×15×19 cm, and the diameter of the discs (101) is in the range of 5 to 20 cm. The distance between each disc is 2 inches for a better yield of BNC. The steel rod (103) on which the discs (101) are mounted is in the range of 25 to 40 cm or about 34 cm in length having 2 to 4 mm or 3 mm threading. The entire chamber with steel rod is connected to the rotation motor (105) with a control panel (109) for controlling the parameters such as rotation speed as well as time. This design is specifically used for 1 Liter media in which the discs are immersed in the nutrient medium. The RDB can be modified as per the scalability of the process up to 10-50 L. The disc dimension can also be varied depending on the outer chamber.

The Rotaspin device is used in the present invention to provide said rotation per minute, however, the same is not limited to this only but may cover any known devices in the art to achieve said rotations per minute.

The water absorbing discs can be made of any suitable absorbent material.

In an embodiment, the water absorbing discs are made of hydrophilic absorbent material.

The rotatory discs can be made of any hydrophilic and water absorbing material for adherence of the bacterial cells on the surface of the rotary discs during fermentation and growth in the nutrient medium. The rotary discs can be made of water absorbing material, for example wood or a hydrophilic polymeric material.

In an embodiment, water absorbing discs are made of wood.

The distance between said discs can be at or above 2 inches. It was found by the inventors of the present invention that the distance between two discs at or above 2 inches worked satisfactorily in the process to provide the high yield of BNC. Whereas the distance between two discs below 2 inches e.g. the distance of 1 inch reduces the yield of BNC compared to 2-inch distance.

Depending on the size of reactor and the length of rod, the number of discs can vary while keeping the distance between discs to be minimum 2 inches.

In an embodiment, the distance between discs is in the range of 2-3 inches. In an embodiment, the discs are of ring shape.

Without bound by any theory the inventors of the present invention believe that the combination of number of days of process, RPM, distance between discs and type of hydrophilic discs used, are the reasons behind higher yields of BNC. Also, it is noted that the hydrophilic nature of discs with ring shape makes the surface sloppier and smoother for better contact with medium liquid at said RPM, producing higher yields of BNC.

The present invention provides an efficient process carried out with Komagataeibacter rhaeticus MCC 0157 and a rotary disc bioreactor for producing BNC with high yield of over 10 gL⁻¹ dry weight basis and in a much shorter duration of within 5 days as compared to the known processes producing lower yield of less than 10 gL⁻¹ dry weight basis while requiring much longer duration of fermentation such as 15 days.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Materials

The nutrient media constituent: glucose, glycerol, yeast extract, peptone, disodium hydrogen phosphate, and citric acid were purchased from Hi Media™ (Mumbai). The steel rod having 3 mm threading of 35 cm length and the rotary discs made of polypropylene and stainless steel of diameter 8 cm were purchased from Pune local market, while wooden disc of 8 cm diameter was purchased from Amazon.com. The outer chamber/reactor was purchased from Amazon.com having the dimension 32×15×19 cm.

### I. Isolation of Bacteria Komagataeibacter rhaeticus

### Example 1

Process of Isolation and Inoculation of Komagataeibacter rhaeticus Bacteria

Isolation of Komagataeibacter rhaeticus from rotten pomegranate fruit was carried out. The isolation method was same as disclosed in the Applicant's co-pending Indian Patent Application number 201911024544. Fruit samples were collected from a local market in Pune, India. Approximately 1 g of sample was incubated in 25 mL of an enrichment medium a Hestrin-Schramm (HS) medium containing: glucose (20 gL⁻¹); yeast extract (5 gL⁻¹); peptone (5 gL⁻¹); disodium hydrogen phosphate (2.7 gL⁻¹); and citric acid (1.15 gL⁻¹) at pH 6.0 supplemented with 100 ppm of cycloheximide to inhibit the growth of yeasts and molds at 28° C. for 10 days. The enriched samples were serially diluted and spread on glucose yeast extract (GYE) agar, and the plates were incubated at 28° C. for 4 days. The colonies which produced a clear zone of solubilization of CaCO₃ on the agar medium were selected and purified. All isolates were purified by repeated-streaking on the HS agar medium, and the purified isolates were either preserved in 20% glycerol and stored at −80° C. or re-streaked on HS agar slants which were stored at 4° C. until further use. The isolates were further subjected to the bacterial cellulose production conditions using the HS liquid media containing glucose (20 gL⁻¹); yeast extract (5 gL⁻¹); peptone (5 gL⁻¹); disodium hydrogen phosphate (2.7 gL⁻¹)); and citric acid (1.15 gL⁻¹), pH 6.0 and incubated for 10-15 days, to select the potential bacteria capable of BC production and providing the inoculant. The formation of pellicles at the air-liquid interface was observed as a positive indication for BC producers. The pellicles formed were of cellulose material, or non-cellulosic (non-cellulosic means the debris in refer to other exopolysaccharide), and this was confirmed by boiling them with 2% NaOH for 30 min. The microfibrillar structures and crystallinity were observed using scanning electron microscopy. The isolated bacteria were identified to be Komagataeibacter rhaeticus and have been deposited under the Budapest treaty at the depository National Centre for Cell Science (NCCS), Pune, Maharashtra, India on 3 Jan. 2019 and have been assigned with accession no. MCC 0157.

### Example 2

Process for Producing Bacterial Nano Cellulose (BNC) by Komagataeibacter rhaeticus MCC 0157 in a Rotary Disc Bioreactor (RDB)

The production of BNC by bacteria K. rhaeticus MCC 0157 was carried out in a 1 L rotary disc bioreactor. The process was carried out by inoculating and fermenting said isolated bacteria Komagataeibacter rhaeticus MCC 0157 of Example 1, such that the discs remained immersed in a nutrient medium containing glycerol in an amount of 3%, yeast extract in an amount of 0.5%, peptone in an amount of 0.5%, disodium hydrogen phosphate in an amount of 0.27%, citric acid in an amount of 0.115%, and rest being water, at pH of 7, under specific speed of RPM of 5, 7, 10 and 15 for a time period of 5, 7, 10 and 15 days with a monitoring interval of 2 to 5 days to obtain bacterial nanocellulose with yield up to 50-70%.

The production of BNC was optimized based on the type of discs, rotation speed of the discs, number of incubation days and the distance between the discs.

Three different types of discs, made of stainless steel, polypropylene and wood were used and evaluated to check their respective maximum production of BNC.

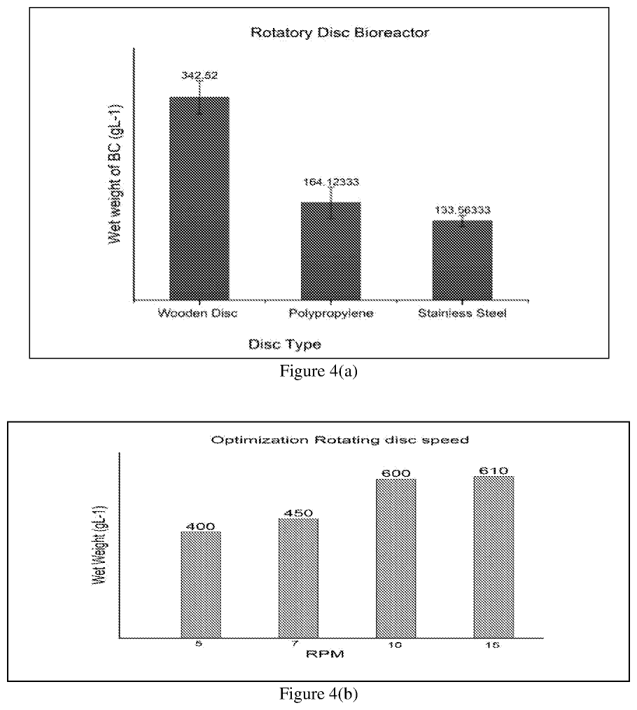

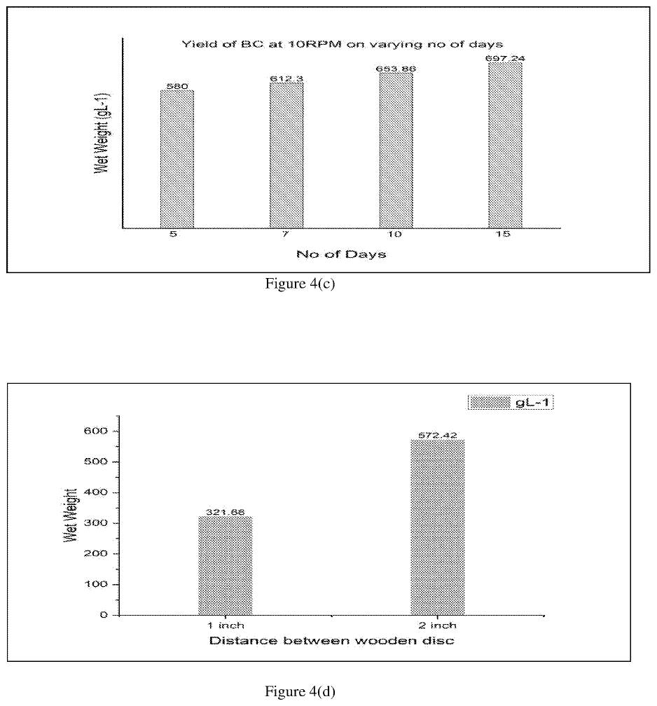

The rotary disc bioreactor with wooden discs showed the maximum yield as compared to polypropylene and stainless-steel discs because the BNC producing bacteria K. rhaeticus MCC 0157 showed more adherence to the wooden disc as compared to polypropylene and stainless steel as depicted in FIG. 4(a). The rotary disc bioreactor with wooden disc showed the yield of 342.52 gL⁻¹ while the rotary disc bioreactor with polypropylene and stainless steel showed the yield of 164.12 gL⁻¹ and 133.56 gL⁻¹ respectively.

As shown in FIG. 4(b), the speed of 10 rpm showed the yield of 600 gL⁻¹ and was selected for further production as 15 rpm showed the yield of 610 gL⁻¹, which was not much of a significant difference than 10 rpm. The number of days for BNC production was also assessed. It was observed that as the number of days increased the yield also increased (FIG. 4(c)). But as shown in Example 3, the production yield was observed to be significant on 5th day itself as compared to the 15th day yield under static conditions. Thus, 5 days incubation was selected with the rotation speed of 10 rpm to carry out the production of BNC in a rotary disc bioreactor.

The distance between each disc was also assessed whether it plays any significant role in the production of BNC. The distance was adjusted as 1 inch and 2 inches. It was observed that the bioreactor with discs placed at 2-inch distance showed significantly higher yield of 572.42 g/L⁻¹ than in bioreactor with discs placed at 1 inch distance producing much less 321 g/L⁻¹ BNC as shown in FIG. 4(d).

### Example 3

Comparison of BNC Production in a Rotary Disc Bioreactor in Accordance with the Present Invention and Static Condition as Per Known Method

The production of BNC by bacteria K. rhaeticus MCC 0157 was carried out in a rotary disc bioreactor in accordance with the present invention and under a static mode for comparison purpose. The process was carried out by inoculating and fermenting said isolated bacteria Komagataeibacter rhaeticus MCC 0157 of Example 1, in a nutrient medium containing glycerol in an amount of 3%, yeast extract in an amount of 0.5%, peptone in an amount of 0.5%, disodium hydrogen phosphate in an amount of 0.27%, citric acid in an amount of 0.115%, and rest being water (quantity sufficient), at pH of 7, under the static condition and in a rotary disc bioreactor with wooden discs placed at a distance of 2 inch and specific speed of RPM of 10 for a time period of 5, 7, 10 and 15 days with a monitoring interval of 2 to 5 days to obtain bacterial nanocellulose.

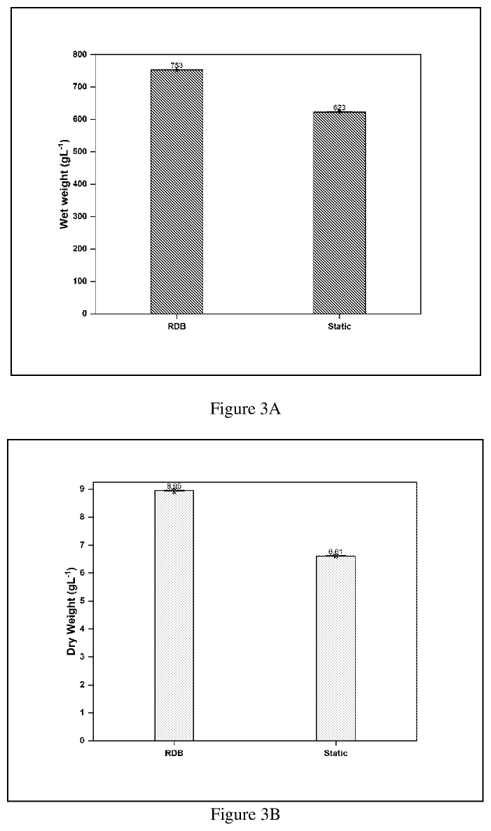

It was observed that the BNC was produced under static condition and in RDB was in the form of a gelatinous membrane. However, fermentation of K. rhaeticus MCC 0157 in a rotary disc bioreactor enhanced the yield in much shorter duration. The organisms adhered to a rotating disc and remained entrapped in the fibrillar network (FIG. 2). The rotary disc bioreactor showed the highest yield compared to static condition fermentation. The production of BNC was found to be 753 gL⁻¹ and 8.95 gL⁻¹ as wet weight and dry weight respectively when produced in the Rotary disc bioreactor, and 623 gL⁻¹ and 6.61 gL⁻¹ wet weight and dry weight respectively, when produced under static culture (FIGS. 3A & 3B).

### Example 4

Production of BNC in RDB with Various Media

The Rotary disc bioreactor with wooden discs was tested for producing BNC with various media used for static fermentation containing different components.

Table 1 shows various media compositions comprising the components selected from glycerol, crude glycerol, sugarcane molasses, yeast extract, peptone, corn steep liquor, Na₂HPO₄, citric acid, and ethanol at varied concentration ranges.

Table 2 shows the average yield of bacterial nanocellulose produced using rotary disc bioreactor as compared to the reported static condition.

TABLE 1
Reported media composition for producing BNC in RDB

| Media | 1 | 2 | 3 | 4 | 5 | 6 | 7 | 8 | 9 |
|---|---|---|---|---|---|---|---|---|---|
| Medium 1 | — | — | 2.0 | 0.5 | 0.5 | — | 0.27 | 0.115 | — |
| Medium 2 | 2.0 | — | — | 0.5 | 0.5 | — | 0.27 | 0.115 | 3.0 |
| Medium 3 | — | — | 2.0 | — | | 0.5 | 0.27 | 0.115 | — |
| Medium 4 | 2.0 | — | — | 0.5 | 0.5 | — | 0.27 | 0.115 | — |
| Medium 5 | — | 2.0 | — | 0.5 | 0.5 | — | 0.27 | 0.115 | — |
| Medium 6 | 2.0 | — | — | — | | 0.5 | 0.27 | 0.115 | — |
| Medium 7 | — | 2.0 | — | — | | 0.5 | 0.27 | 0.115 | — |

Medium ingredient (%)—1: Glycerol, 2: Crude Glycerol, 3: Sugarcane molasses, 4: Yeast Extract, 5: Peptone, 6: Corn steep liquor, 7: Na₂HPO₄, 8: Citric Acid, 9: Ethanol.

TABLE 2
BNC produced with various media in RDB and static condition

| | | | | Average | | Dry wt. | |
|---|---|---|---|---|---|---|---|
| | | | | wet wt. | Dry wt. | Reported | |
| | Yield Wet Wt. | | | Present | Present | Static | |
| | Present invention | | | invention | invention | condition | |
| Media | (g/L) | | | (g/L) | (g/L) | (g/L) | Reference* |
| Medium 1 | 323.4 | 298.46 | 314 | 311.95 | 6.03 | 1.2 | Jung et al., J. |
| | | | | | | | Chem. |
| | | | | | | | Eng. 2007, 24, |
| | | | | | | | 265. |
| Medium 2 | 398 | 501.32 | 433.11 | 444.14 | 8.58 | 7 | Krystynowicz |
| | | | | | | | et al., |
| | | | | | | | Biotechnol. |
| | | | | | | | 2002, 29, |
| | | | | | | | 189 |
| Medium 3 | 328.62 | 311.16 | 297.14 | 312.30 | 6.03 | 1.2 | Bae & |
| | | | | | | | Shoda., |
| | | | | | | | Bioeng. 2005, |
| | | | | | | | 90, 20 |
| Medium 4 | 701 | 724.62 | 714 | 713.20 | 13.78 | 7.2 | Castro et al., |
| | | | | | | | Polym. 2011, |
| | | | | | | | 84, 96 |
| Medium 5 | 550 | 598.71 | 567.1 | 571.93 | 11.05 | 4.8 | Wu & Liu., |
| | | | | | | | Carbohydr. |
| | | | | | | | Polym. 2012, |
| | | | | | | | 90, 116 |
| Medium 6 | 311.83 | 321.62 | 310.12 | 314.52 | 6.08 | 1.75 | Shah et al., 2 |
| | | | | | | | Carbohydr. |
| | | | | | | | Polym |
| | | | | | | | 2013, 98, |
| | | | | | | | 1585 |
| Medium 7 | 112 | 141 | 98 | 117 | 2.26 | 1 | Krystynowicz |
| | | | | | | | et al., |
| | | | | | | | Bielecki, J. |
| | | | | | | | Ind. |
| | | | | | | | Microbiol. |
| | | | | | | | Biotechnol. |
| | | | | | | | 2002, 29, |
| | | | | | | | 189 |

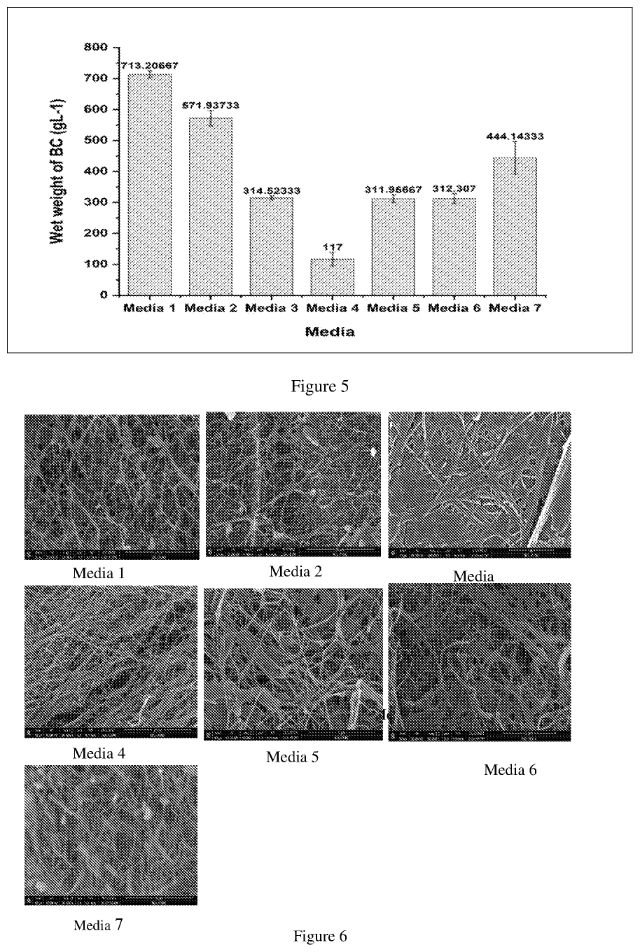

As can be seen from the above Table 2 and FIG. 5 the yield of BNC produced in accordance with the present invention in a RDB was significantly higher as compared to the BNC produced under the static conditions using various reported media. There was approximately a 2-fold increase in the dry weight yield of the BNC produced in RDB as compared to the BNC produced under static condition, thus proving that the RDB enhances the yield effectively. FIG. 6 shows FESEM images revealing the fibrillar and non-woven arrangement of bacterial nanocellulose from RDB using different media.

### III. Characterization of BNC Produced by RDB and Static Conditions

### Example 5

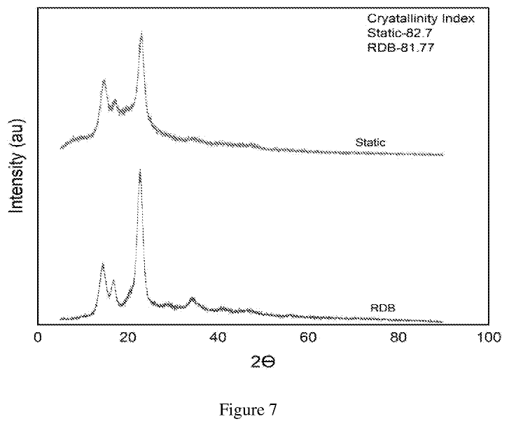

The X-ray diffractogram (XRD) analysis was carried out for BNC produced under the static condition and in accordance with the present invention in RDB. The XRD patterns obtained showed the characteristics of cellulose peaks at 2Θ angles z 14°, 16° and 22° in both the conditions (FIG. 7).

### Example 6

The crystallinity index of the membranes of BNC produced under the static condition and in accordance with the present invention in RDB were calculated using the Segal method (peak height). The crystallinity index recorded for the BNC obtained from static condition was 82.7 and the crystallinity index obtained for the BNC produced in accordance with the present invention in the RDB was 81.77 confirming the cellulose produced by both the method.

### Example 7

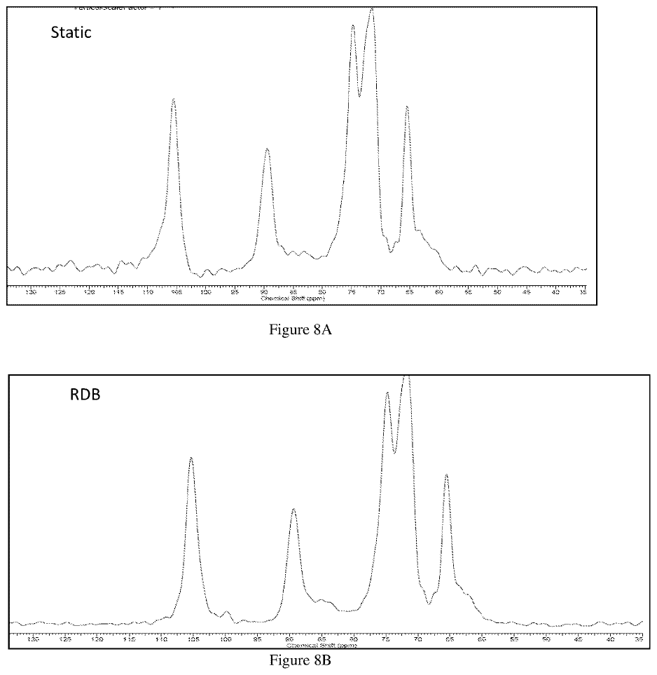

¹³C NMR spectra for BNC produced under the static condition and in accordance with the present invention in RDB are shown in FIGS. 8A and 8B respectively. All the resonance lines were assigned for C-1 to C-6. Both spectra are very similar and show resonance lines at 102-108 ppm, 81-93 ppm and 60-68 ppm which were assigned to the C-1, C-4 and C-6 carbons, respectively. The clusters of resonances from 70 ppm to 80 ppm were assigned to the C-2, C-3, and C-5 carbons. It is known that nano cellulose is composed of two distinct crystalline structures i.e., Ia and Ib. The fractions of both allomorphs vary depending on the source of nanocellulose. Mostly, nanocellulose from bacteria and valonia is Ia rich, whereas plant-based nanocellulose is rich in the Ib crystalline phase. Due to the presence of smaller doublets beside the prominent central resonance line, C-1 and C-4 appear to be triplets, indicating the presence of a minor fraction of the Ib phase and a major fraction of the Ia phase of nanocellulose. Here, Ia and Ib are the crystalline structures found in cellulose i.e., Ia is triclinic structure and Ib is monoclinic structure thus are nanocellulose contains the mixture of these two crystalline structures.

TABLE 3
¹³C NMR. C-1 to C-6 values of static and RDB BNC samples

| Carbon group | RDR (ppm value) | Static (ppm value) |
|---|---|---|
| C1 | 102-108 | 102-108 |
| C2 | 70-80 | 70-80 |
| C3 | 70-80 | 70-80 |
| C4 | 81-93 | 81-93 |
| C5 | 70-80 | 70-80 |
| C6 | 60-68 | 60-68 |

### Example 8

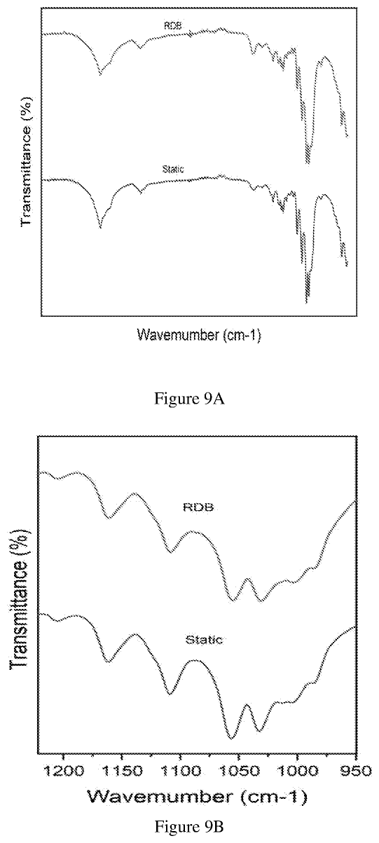

The structural analyses of membranes of BNC produced under the static condition and in accordance with the present invention in RDB were carried out by FTIR analysis. The FTIR spectra obtained for the membranes of BNC produced under the static condition and in accordance with the present invention in RDB are in the range of 4000-500 cm⁻¹ and are represented in FIGS. 9A and 9B respectively. Both spectra obtained showed the presence of characteristic bands known for nanocellulose I. The sharp peaks at position 3344 cm⁻¹ and 3242 cm⁻¹ show the intermolecular hydrogen bonding which are typical peaks of —OH stretching vibrations, indicating the properties of nanocellulose I. Absence of bands in the regions of 3488 cm⁻¹ and 3447 cm⁻¹ (OH stretching due to intramolecular hydrogen bonding) confirmed the absence of the nanocellulose II properties. The signature peak for nanocellulose I at 1430 cm⁻¹ (a symmetric CH2 bending vibration) further confirmed the property nanocellulose I in the structure.

The complex fingerprint regions ranging from 1800-800 cm⁻¹ showed the following characteristic bands; 1651 cm⁻¹ (water absorbed), 1427 cm⁻¹ (CH2 symmetric bending), 1370 cm⁻¹ (CH bending), 1337 cm⁻¹ (OH in-plane bending), 1315 cm⁻¹ (CH2 wagging), 1280 cm⁻¹ (CH bending), 1161 cm⁻¹ (C—O—C asymmetric stretching at the β-glycosidic linkage), 1108 cm⁻¹ (C—O bond stretching), 1054 cm−1 (C—O—C ring skeletal vibration), and 896 cm⁻¹ (weak and broad bands of a b-linked polymer). The given FTIR spectra revealed the existence of both the Ia and Ib allomorphs in static and RDB, which are in agreement with the NMR results. The peaks at 3220 cm⁻¹ and 750 cm⁻¹, and 3283 cm⁻¹ and 710 cm⁻¹, have been assigned to the triclinic allomorph Ia and the monoclinic allomorph Ib respectively. Here, the nanocellulose exists in two allomorphic forms i.e., I and II.

TABLE 4
FTIR spectra data of BNC produced under
the static condition and in RDB

| | Wavenumber | Wavenumber |
|---|---|---|
| | cm⁻¹ | cm⁻¹ |
| Functional group | (RDB) | (Static) |
| OH stretching vibration | 3344 | 3344 |
| | 3242 | 3242 |
| Water absorbed | 1651 | 1651 |
| CH₂ symmetric bending | 1427 | 1427 |
| CH bending | 1370 | 1370 |
| OH in plane bending | 1337 | 1337 |
| CH₂ wagging | 1351 | 1351 |
| CH bending | 1280 | 1280 |
| (C—O—C asymmetric stretching | 1161 | 1161 |
| at the β-glucosidic linkage | | |
| C—O bond stretching | 1108 | 1108 |
| C—O—C ring skeletal vibration | 1054 | 1054 |
| weak and broad bands of a b- | 896 | 896 |
| linked polymer | | |

### Example 9

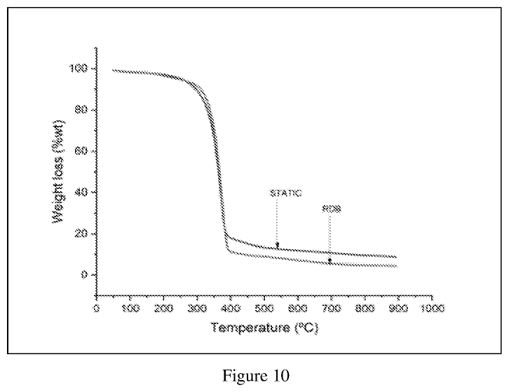

The thermal degradation behavior patterns of the membranes of BNC produced under the static condition and in accordance with the present invention in RDB are shown in FIG. 10. Both curves showed the same thermal properties with two main weight losses. The initial minor weight loss curve observed was associated with the evaporation of surface water. At a temperature around 270° C., a second weight loss began, which corresponds to the depolymerization and degradation into water and gases. Maximum degradation of weight loss i.e., 85 and 95% was observed up to 395° C. for BCN produced under both static and RDB conditions respectively.

### Advantages of the Invention

Novel approach for obtaining improved yield of BNC in shorter (over 10 grams per liters in just 5 days).
The disclosed process in cost effective.
This approach can be scalable to industrial level and depending on the application the process helps to produce high yield of BNC in lesser number of days.

The invention claimed is:

1. A process for production of bacterial nanocellulose (BNC), the process comprising:
inoculating bacteria Komagataeibacter rhaeticus MCC 0157 to a nutrient medium in a rotatory disc bioreactor; and
fermenting at a speed of 5-15 RPM for a time period of 5-10 days, wherein the rotatory disc bioreactor comprises at least two circular rotatory discs of hydrophilic absorbent material placed at a predetermined distance in a range of 2-3 inches between them.

2. The process of claim 1, wherein the nutrient medium is:
modified Hestrin-Schramm (HS) liquid medium comprising glycerol in a weight range of 2-3%;
yeast extract in a weight range of 0.1-2.0%;
peptone in a weight range of 0.1-2.0%;
disodium hydrogen phosphate in a weight range of 0.1-1.0%; citric acid in a weight range of 0.1-1.0%; and
water in sufficient quantity.

3. The process of claim 1, wherein a pH of the nutrient medium is maintained in a range of 6 to 7.

4. The process of claim 1, wherein the fermentation in the rotatory disc bioreactor is carried out at a speed selected from a group consisting of 5, 7, 10, and 15 RPM.

5. The process of claim 1, wherein the circular rotatory discs of the bioreactor are immersed in the nutrient medium.

6. The process of claim 1, wherein the production of bacterial nanocellulose (BNC) is monitored at an interval of 2 to 5 days; and
adding additional media in case of complete utilization of the media to monitor the yield of the BNC for subsequent days till 5 to 10 days.

7. The process of claim 1, wherein the fermentation is carried out for 5 days.

8. A rotary disc bioreactor comprising:
a plurality of water absorbing disc with pre-defined distance between them and arranged to be immersed in a nutrient medium,
a rod for holding said discs,
a rotation motor and a control panel connected to the said rod, and
an outer chamber for covering or protecting said rod and discs.

9. The rotary disc bioreactor of claim 8, wherein the water absorbing discs are made of a hydrophilic absorbent material.

10. The rotary disc bioreactor of claim 8, wherein the water absorbing discs are made of wood; and wherein the distance between the discs is 2-3 inches.
